(19) United States
(12) Patent Application Publication
(10) Pub. No.: US 2023/0213520 A1
(43) Pub. Date: Jul. 6, 2023

(54) IN VITRO METHOD FOR THE PROGNOSIS OF DISEASE PROGRESSION IN A PATIENT THAT SUFFERS FROM OR IS AT RISK OF DEVELOPING A SOLID TUMOR

(71) Applicant: Bergonié Institute, Bordeaux (FR)

(72) Inventors: Antoine ITALIANO, Bordeaux (FR); Sophie COUSIN, Bordeaux (FR)

(21) Appl. No.: 17/925,291

(22) PCT Filed: May 12, 2021
(86) PCT No.: PCT/EP2021/062705

§ 371 Date: Nov. 14, 2022

(30) Foreign Application Priority Data
May 13, 2020 (EP) 20174467.9

(51) Int. Cl. G01N 33/574

(52) U.S. Cl. CPC G01N 33/57419; G01N 2333/70517; G01N 2333/70596; G01N 2800/52

(57) ABSTRACT

The present invention provides an in vitro method for the prognosis of disease progression in a patient that suffers from or is at risk of developing a solid tumor, said method comprising determining, in one or more samples from said patient, at least one of intratumoral infiltration by macrophages, and/or proximity of lymphocytes to tumor cells, and/or proximity of macrophages to tumor cells, and/or presence or absence of one or more nearby tertiary lymphoid structures (TLS), and an anti-angiogenic drug, optionally combined with an immune checkpoint inhibitor, for use in the treatment of such patient.

### FIELD OF THE INVENTION

The present application relates to an in vitro method for the prognosis of disease progression in a patient that suffers from or is at risk of developing a solid tumor.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

### INTRODUCTION

Cancers of the digestive system, including colorectal cancer (CRC) and its metastatic variant remains one of the leading causes of cancer-related deaths worldwide. In the metastatic setting, the standard of care for patients who are not candidate for surgical procedures, is based on palliative fluorouracil-based chemotherapy regimens associated with agents targeting angiogenesis or the epidermal growth factor receptor.

One approach for treating these modalities is the use of an anti-angiogenic drug, also called angiogenesis inhibitor. These drugs inhibit the growth of blood vessels (angiogenesis). In the context of the invention, a tumor can stimulate the growth of new vessels to expand the tumor to other organs and improve its vascularization. Thus, inhibiting angiogenesis may reduce or stop the growth of tumors.

There currently two classes of anti-angiogenic drugs, namely tyrosin kinase inhibitor, and binders, inhibitors or antagonists of VEGF, FGF, PGF, PDGF, VEGFR, FGFR, PGFR or PDGFR.

One such anti-angiogenic drug, Regorafenib, a small molecular targeting several protein kinases involved in tumor angiogenesis, including angiogenic, stromal and oncogenic receptor tyrosine kinase (RTK), is the sole targeted therapy approved for the management of metastatic CRC patients who have failed standard chemotherapies and had no other treatment options. This approval was based on the result of a pivotal randomized study which showed that patients who received Regorafenib in addition to supportive care experienced longer progression-free survival (PFS) (median of 2 months vs. 1.7 months) and overall survival (OS) than those who received placebo (median of 6.4 months vs. 5 months), despite an objective response rate which was only 1%. This clinical benefit appears as modest and therefore new therapeutic strategies are needed to improve outcome of chemo-refractory CRC patients.

However, Regorafenib has been withdrawn inter alia from the from the German market after the healthcare reimbursement agency had judged that an additional benefit of Regorafenib for patients with metastatic colorectal cancer compared to best possible supportive treatment would not be proven.

This withdrawal leaves an important medical need unanswered—namely, inter alia, the management of metastatic CRC patients who have failed standard chemotherapies and had no other treatment options. Yet also for cancers of other digestive organs, including colon cancer, metastatic colon cancer, esophageal cancer, metastatic esophageal cancer, gastric cancer and metastatic gastric cancer, the withdrawal of Regorafenib reduces the therapeutic options.

Hence, it is one object of the present invention to provide means and methods to enhance the therapeutic options for patients suffering from or being diagnosed for a cancer of a digestive organ.

It is one other object of the present invention to provide means and methods to expand the therapeutic spectrum of anti-angiogenic drugs including, yet not limited to, Regorafenib.

These and other objects are solved by the subject matter of the present invention.

### BRIEF DESCRIPTION OF THE DRAWINGS

Table 1. Characteristics of patients that were enrolled in the study FIG. 1. Flow-chart of patients included in the study.

FIG. 2: Waterfall plot (A) and spider plot (B) of best overall response in microsatellite stable colorectal cancer patients treated with Regorafenib plus Avelumab (n=40, Response based on central review assessment according to RECIST 1.1).

FIG. 3. Kaplan-Meier curves of progression-free survival (A) and overall survival (B) in microsatellite stable colorectal cancer patients treated with Regorafenib and avelumab

FIG. 4: Kaplan-Meier curves of progression-free survival in microsatellite stable colorectal cancer patients treated with Regorafenib and avelumab according to the density of tumoral CD163+ macrophages considering a cut-off value of 149,83 cells/mm2

FIG. 5: Kaplan-Meier curves of progression-free survival in microsatellite stable colorectal cancer patients treated with Regorafenib and avelumab according to the density of tumoral CD163+ macrophages considering a cut-off value of 200 cells/mm2

FIG. 6: Kaplan-Meier curves of progression-free survival in microsatellite stable colorectal cancer patients treated with Regorafenib and avelumab according to the distance between CD8+ cells and Keratin+ cells considering a cut-off value of 107,94 μm

FIG. 7: Kaplan-Meier curves of progression-free survival in microsatellite stable colorectal cancer patients treated with Regorafenib and avelumab according to the distance between CD8+ cells and Keratin+ cells considering a cut-off value of 70 μm.

FIG. 8: Kaplan-Meier curves of progression-free survival in microsatellite stable colorectal cancer patients treated with Regorafenib and avelumab according to the distance between CD8+ cells and Keratin+ cells considering a cut-off value of 70 μm and the density of intratumoral CD163+ cells considering a cut-off value of 200 cells/mm²

FIG. 9: Illustration of image analysis approach after immunostaining with respective antibodies (see text), showing the different steps of i) tissue segmentation—tumor vs stroma vs others, ii) cell segmentation allowing the segmentation into nucleus, cytoplasm and membrane compartments for each cells whatever the region and iii) phenotyping of cells including tumor cells based on keratin staining, effector T cells based on CD8 positivity and tumor associated macrophages

PanKer=PanKeratin antibody, to identify presence of keratin (tumor marker) and hence differentiate tumor tissue from stroma. CD163=anti CD163 antibody, to identify macrophages and determine intratumoral infiltration thereof and proximity thereof to tumor cells. CD8=anti CD8 antibody, to identify lymphocytes and determine proximity thereof to tumor cells

Table 2: overview of different targets that can be measured in the context of the present invention

### SUMMARY OF THE INVENTION

According to one aspect of the invention, an in vitro method for the prognosis of disease progression in a patient that suffers from or is at risk of developing a solid tumor is provided, said method comprising determining, in one or more samples from said patient, at least one of

a) intratumoral infiltration by macrophages, and/or
b) proximity of lymphocytes to tumor cells, and/or
c) proximity of macrophages to tumor cells, and/or
d) presence or absence of one or more nearby tertiary lymphoid structures (TLS)
wherein the prognosis is an assessment regarding the likelihood of efficacy of a therapy comprising administration of an anti-angiogenic drug.

As used herein, the term “intratumoral infiltration” describes a condition where cells, in particular immune cells, have left the bloodstream and migrated towards a tumor.

As used herein, the term “proximity” describes a relative or absolute distance between the respective cell types, preferably expressed as mean, average or median distance.

As used herein, the term “macrophages” relates to a type of white blood cells of the immune system that engulfs and digests cellular debris, foreign substances, microbes, cancer cells, and anything else that does not have the type of proteins specific to healthy body cells on its surface in a process called phagocytosis. In one embodiment, the macrophages are characterized by CD68 expression and/or CD163 expression.

As used herein, the term “lymphocytes” relates to a type of white blood cells of the immune system. Lymphocytes include natural killer cells, which function in cell-mediated, cytotoxic innate immunity), T cells (for cell-mediated, cytotoxic adaptive immunity), and B cells (for humoral, antibody-driven adaptive immunity). In one embodiment, the macrophages are characterized by CD8.

As used herein, the term TLS relates to one or more ectopic lymphoid organs that develop in non-lymphoid tissues at sites of chronic inflammation including tumors.

As used herein, the term “nearby” suggests that the TLS is located intra-tumoral, in peritumoral regions, and/or at the invasive front of the tumor. As used herein, the term “stroma” or “tumor stroma” refers to the connective tissue framework and non-tumor cells of a tumor. Examples of some non-tumor cells found in a tumor stroma are fibroblasts and endothelial cells. These cells produce the sum extracellular matrix (ECM) of the tumor. Sometimes, the ECM and the non-malignant cells of the tumor are defined as the “tumor stroma”

As used herein, the term “tumor” means a mass of transformed cells that are characterized, at least in part, by containing angiogenic vasculature. The transformed cells are characterized by neoplastic uncontrolled cell multiplication which is rapid and continues even after the stimuli that initiated the new growth has ceased.

As used herein, the term “metastases” or “metastatic tumor” refers to a secondary tumor that grows separately elsewhere in the body from the primary tumor and has arisen from detached, transported cells, wherein the primary tumor is a solid tumor. The primary tumor, as used herein, refers to a tumor that originated in the location or organ in which it is present and did not metastasize to that location from another location.

In one embodiment, at least the intratumoral infiltration by macrophages, and the proximity of lymphocytes to tumor cells are determined. In one embodiment, at least the intratumoral infiltration by macrophages, and the proximity of macrophages to tumor cells are determined. In one embodiment, at least the intratumoral infiltration by macrophages, and the presence or absence of one or more nearby tertiary lymphoid structures (TLS) are determined.

In one embodiment, at least the proximity of lymphocytes to tumor cells, and the proximity of macrophages to tumor cells are determined. In one embodiment, at least the proximity of lymphocytes to tumor cells, and the presence or absence of one or more nearby tertiary lymphoid structures (TLS) are determined. In one embodiment, at least the proximity of macrophages to tumor cells, and the presence or absence of one or more nearby tertiary lymphoid structures (TLS) are determined. In one embodiment, the intratumoral infiltration by macrophages and the proximity of lymphocytes to tumor cells are determined. In one embodiment, the intratumoral infiltration by macrophages and the proximity of lymphocytes to tumor cells and the proximity of macrophages to tumor cells are determined.

In one embodiment, at least two of the intratumoral infiltration by macrophages and the proximity of lymphocytes to tumor cells and the proximity of macrophages to tumor cells are determined, and optionally the proximity of lymphocytes to tumor cells is determined.

According to one embodiment, the prognosis is an assessment regarding the likelihood of efficacy of a combination therapy comprising,

(i) administration of an anti-angiogenic drug, and
(i) administration of an immune checkpoint inhibitor,
wherein the combination of the two drugs is administered to a patient concomitantly or consecutively.

As used herein, the term “anti-angiogenic drug”, also called “angiogenesis inhibitor” refers to a substance that inhibits the growth of blood vessels (angiogenesis). In the context of the invention, a tumor can stimulate the growth of new vessels to expand the tumor to other organs and improve its vascularization. Thus, inhibiting angiogenesis may reduce or stop the growth of tumors. Inhibiting angiogenesis requires treatment with anti-angiogenic factors, or drugs which reduce the production of pro-angiogenic factors, preventing receptor binding or blocking their actions.

As used herein, the term “immune checkpoint inhibitor” refers to any compound inhibiting the function of an immune inhibitory checkpoint protein—i.e., a protein, like a cytokine or ligand or receptor, that is capable to turn down an immune response, e.g., a cytolytic or apoptotic T cell attack. Inhibition includes reduction of function and full blockade. A number of immune checkpoint inhibitors are known and disclosed elsewhere herein, and in analogy of these known immune checkpoint protein inhibitors, alternative immune checkpoint inhibitors may be developed in the future.

The development of immune checkpoint inhibitors has dramatically changed the landscape of treatment of several cancers—in particular, malignant melanoma and non—small cell lung cancer. However, in CRC, only patients with the subset of mismatch-repair-deficient or microsatellite instability-high (dMMR/MSI-H) tumors have been shown to respond to treatment with immune checkpoint inhibitors when used as single agent.

The inventors have shown significant patient advantage in terms of overall survival (OS) and progression free survival in patents suffering from, or being diagnosed for, a solid tumor, in particular a tumor of a digestive organ or its metastases when being treated with such combination.

The inventors have hypothesized on possible explanation for their findings. They realized that e.g., inhibition of VEGR may inhibit T-regulatory cell proliferation in colorectal cancer patients. Furthermore, they understood that VEGF-A which is abundant in the tumor microenvironment of human tumors has been shown to upregulate the expression of PD-1, i.e., an immune checkpoint, which plays a crucial role in in CD8+ T cell exhaustion.

Further, they realized that the tyrosin kinase inhibitor Regorafenib inhibits CSF 1R, a tyrosine kinase receptor that is involved in macrophage proliferation.

Therefore, there seem indeed to be theories that may help to explain the observed synergistic effect of co-administration of an anti-angiogenic drug, and an immune checkpoint inhibitor.

According to one embodiment, the solid tumor is a tumor of a digestive organ.

According to one embodiment, the solid tumor is selected from the group consisting of colon cancer, metastatic colon cancer, colorectal cancer or metastatic colorectal cancer, esophageal cancer, metastatic esophageal cancer, gastric cancer and metastatic gastric cancer.

According to one embodiment, the sample from the patient has been derived from at least one selected from the group consisting of liver biopsy, lung biopsy, colon biopsy, gastric biopsy, esophageal biopsy and/or colorectal biopsy.

It is important in this respect that in particular colorectal and colon carcinoma have a high likelihood to be the source of metastases in the liver and/or the lung.

In one embodiment, the sample from the patient is a tissue slice. In one embodiment, the sample from the patient is a biopsy. In one embodiment, the sample from the patient is a smear sample. In one embodiment, the sample from the patient is a Fine-needle aspiration (FNA), or sampling (FNS)

In one embodiment, the sample from the patient is a fresh sample. In one embodiment, the sample from the patient is a frozen sample. In still one embodiment, the sample from the patient is an FFPE preserved sample.

According to one embodiment, the in vitro method further comprises the step of determining, in the sample, the presence or absence of stroma tissue and/or tumor tissue.

This can be done, for example, by methods comprising an immunoassay and/or methods comprising PCR and/or nuclei acid hybridization.

In general, all these methods are well known to the skilled person, and their implementation requires routine knowledge and expertise.

As used herein, the term “a method comprising an immunoassay” relates to a method where an immunological molecule, e.g., an antibody, or a protein having comparable target specific binding characteristics, is used to detect the presence of a target molecule.

In one embodiment, said method comprises IHC (Immunohistochemistry assay), ICC (Immunocytochemistry assay), IF (Immunofluorescence assay) or IHFC (Immunohistofluorescence assay).

As used herein, the term “immunostaining and subsequent image analysis” relates to immunoassay methods where the target is stained by means of the immunological molecule or the protein having comparable target specific binding characteristics, namely by direct or indirect labelling of the molecule or protein, and subsequent analysis of the sample by means of video microscopy or other imaging means, and subsequent digital image processing.

As used herein, the term “a method comprising PCR” relates to a method where polymerase chain reaction is used to detect the presence or absence of a nucleic acid (DNA or mRNA) encoding for a target molecule.

In one embodiment, the method comprising PCR and/or nuclei acid hybridization comprises at least one of
Fluorescent in situ hybridization (FISH),
In situ PCR,
Real time PCR, and/or
Reverse transcription PCR/qPCR

All these methods are well known to the skilled person. While the former two deliver spatial resolution and can hence be used for density or proximity measurements, the latter two don't and can hence only be used for infiltration measurements, as e.g. of macrophages in to the tumor tissue.

In one embodiment, the tumor tissue in the sample is identified by detecting the presence or absence of a tumor marker.

In one embodiment, one such tumor marker is a keratin, preferably a cytokeratin.

Cytokeratins are keratin proteins found in the intracytoplasmic cytoskeleton of epithelial tissue. They are an important component of intermediate filaments, which help cells resist mechanical stress. Expression of these cytokeratins within epithelial cells is largely specific to particular organs or tissues. Thus they can be used as markers to identify tumor tissue. In another embodiment, one such tumor marker is PSA (prostate specific antigen) or CA 19-9.

This can be done, in one embodiment, by methods comprising an immunoassay, e.g., with the use of an anti-keratin antibody, preferably an anti-cytokeratin antibody.

Preferably, such method comprises immunostaining and subsequent image analysis.

The table shows some tumor markers (with CK standing for cytokeratin), their meaning and suitable antibodies for detection.

| target | for the detection of . . . | target can be detected e.g. with |
|---|---|---|
| Pan | all types of tumors | Pan Keratin antibody (clone |
| Keratin | | AE1/AE3/PCK26, Roche) |
| CK 20 | Biliary duct/Colon/ | anti-Cytokeratin 20 antibody (Abcam |
| | Merkel cell tumor | [EPR1622Y] (ab76126)) |
| CK7 | Biliary duct/Bladder/ | Cytokeratin 19 (SP52) - Roche |
| | Breast/Lung/Ovary/ | |
| | Pancreas/Uterus tumor | |
| CK8/18 | Biliary duct/Bladder/ | anti-cytokeratin CK8/18 (5D3) - Leica |
| | Breast/Colon/Kidney/ | |
| | Liver/Lung/Ovary/ | |
| | Pancreas/Pleura/ | |
| | Prostate/Merkel cell/ | |
| | Stomach/Uterus tumor | |
| CK19 | Biliary duct/Bladder/ | Cytokeratin 19 (A53- B/A2.26) - Roche |
| | Breast/Cervix/Colon/ | |
| | Lung/Ovary/Pancreas/ | |
| | Pleura/Prostate/ | |
| | Stomach/Uterus tumor | |
| PSA | Prostrate tumor | anti-Prostate Specific Antigen antibody |
| | | (Abcam ab53774) |
| CA19-9 | Pancreas tumor | CA19-9 Antibody (MA5-13275) by |
| | | Thermo Fisher |

One or more of these tumor markers can advantageously be combined.

The skilled person is capable of finding, based on the information provided herein and routine considerations, other suitable antibodies that can be used to detect tumor tissue, in particular tumor markers, in particular keratin proteins or cytokeratin proteins.

In one embodiment, the stroma tissue in the sample is identified as the part of the sample that does not show a tumor marker. In one embodiment, the stroma tissue in the sample is identified as part of the tissue characterized by absence of tumor marker. In one embodiment, the absence of a tumor marker is the absence of keratin, preferably absence of a cytokeratin.

In one embodiment, the stroma tissue in the sample is identified by detecting the presence or absence of a stroma marker. In one embodiment, such stroma marker is endostatin. Endostatin is the C-terminal fragment of the BM protein type XVIII collagen.

According to one embodiment, the anti-angiogenic drug is at least one of
a tyrosin kinase inhibitor, and/or
a binder, inhibitor or antagonist of at least one of VEGF, FGF, PGF, PDGF, VEGFR, FGFR, PGFR or PDGFR.

Herein, VEGF includes VEGF-A, VEGF-B, VEGF-C and VEGF-D, and VEGFR includes receptors thereof.

In embodiments according to the invention, the binder, inhibitor or antagonist is an antibody, or a target binding fragment or derivative thereof.

As used herein, the term “derivative” encompasses, inter alia, receptor Fc-fusion proteins (so called “-cept” proteins).

As used herein, the term “fragment” encompasses, inter alia, Fab fragments, scFv fragment, (Fab)2 fragment, diabodies, and the like.

According to one embodiment, the antibody is at least one selected from the group consisting of Bevacizumab, Ramucirumab Ziv-aflibercept, or Ranibizumab

In one embodiment, the tyrosin kinase inhibitor is at least one selected from the group consisting of Afatinib, Alectinib, Axitinib, Bosutinib, Cabozantinib, Crizotinib, Dasatinib, Erlotinib, Gefitinib, Gilteritinib, Ibrutinib, Imatinib, Lapatinib, Lenvatinib, Midostaurin, Neratinib, Nilotinib, Nintedanib, Pazopanib, Ponatinib, Regorafenib, Ruxolitinib, Sorafenib, Sunitinib, Tofacitinib, and Vandetanib.

In one embodiment the tyrosin kinase inhibitor is Regorafenib. As discussed elsewhere herein, Regorafenib is an oral multi-kinase inhibitor which targets angiogenic, stromal and oncogenic receptor tyrosine kinase (RTK). It has obtained regulatory approval by the EMA and the FDA, but has been withdrawn inter alia from the from the German market after the healthcare reimbursement agency had judged that an additional benefit of Regorafenib for patients with metastatic colorectal cancer compared to best possible supportive treatment would not be proven. However, the inventors of the present invention have shown a benefit of Regorafenib for patients suffering form or being diagnosed for solid tumors (in particular those of a digestive organ) and stratified as disclosed herein

According to one embodiment, the immune checkpoint inhibitor is a binder, inhibitor or antagonist of at least one of CTLA-4, PD-1, PD-L1, LAG 3, TIM3, OX40, and/or TIGIT.

In embodiments according to the invention, the binder, inhibitor or antagonist is an antibody, or a target binding fragment or derivative thereof.

According to one embodiment, the antibody is at least one selected from the group consisting of is at least one selected from the group consisting of Ipilimumab (anti-CTLA-4), Nivolumab (anti-PD-1), Pembrolizumab (anti-PD-1), Cemiplimab (anti-PD-1), Spartalizumab (anti-PD-1), Atezolizumab (anti-PD-L1), Avelumab (anti-PD-L1), Durvalumab (anti-PD-L1), Etigilimab (anti-TIGIT), BGB-A1217 (anti-TIGIT) BMS-986207 (anti-TIGIT), AB154 (anti-TIGIT) ASP8374 (anti-TIGIT), MK 7684 (anti-TIGIT), and/or Tiragolumab (anti-TIGIT)

In one embodiment, the immune checkpoint inhibitor is a binder, inhibitor or antagonist of PD-L1. In one embodiment the immune checkpoint inhibitor is Avelumab (PD-L1). Avelumab is a fully human monoclonal antibody which targets the protein programmed death-ligand 1 (PD-L1). It has received orphan drug designation by the European Medicines Agency (EMA) for the treatment of gastric cancer. The US Food and Drug Administration (FDA) approved it in March 2017 for Merkel-cell carcinoma. The EMA approved it in September 2017 for the same indication. The inventors of the present invention have shown a benefit of Avelumab for patients suffering form or being diagnosed for solid tumors and stratified as disclosed herein.

In one embodiment, the prognosis according to the present invention is an assessment regarding the likelihood of efficacy of a combination therapy comprising,

(i) administration of an anti-angiogenic drug, preferably a tyrosin kinase inhibitor, preferably Regorafenib, and
(ii) administration of an immune checkpoint inhibitor a binder, preferably an inhibitor or antagonist of at least one of CTLA-4, PD-1, PD-L1, LAG 3, TIM3, OX40, and/or TIGIT,
wherein the combination of the two drugs is administered to a patient concomitantly or consecutively.

In one embodiment, the prognosis according to the present invention is an assessment regarding the likelihood of efficacy of a combination therapy comprising,

(i) administration of an anti-angiogenic drug, preferably a tyrosin kinase inhibitor or a binder, inhibitor or antagonist of at least one of VEGF, FGF, PGF, PDGF, VEGFR, FGFR, PGFR or PDGFR, and
(ii) administration of a binder, inhibitor or antagonist of at least one of CTLA-4, PD-1, PD-L1, LAG 3, TIM3, OX40, and/or TIGIT, preferably of Avelumab
wherein the combination of the two drugs is administered to a patient concomitantly or consecutively.

In one embodiment, the prognosis according to the present invention is an assessment regarding the likelihood of efficacy of a combination therapy comprising,

(i) administration of Regorafenib, and
(ii) administration of Avelumab
wherein the combination of the two drugs is administered to a patient concomitantly or consecutively.

According to one embodiment, in the in vitro method,
a) the intratumoral infiltration by macrophages, is determined by at least one of a method comprising an immunoassay or a method comprising PCR
b) the proximity of lymphocytes to tumor cells is determined by a method comprising an immunoassay
c) the proximity of macrophages to tumor cells is determined by a method comprising an immunoassay and/or
d) presence or absence of one or more nearby tertiary lymphoid structures (TLS) is determined by a method comprising an immunoassay.

Preferably, such immunoassay method comprises immunostaining and subsequent image analysis.

The proximity of different cells to one another and/or the density of a given cell type in a given sample can be carried out by a method comprising an immunoassay. Preferably, such immunoassay method comprises immunostaining and subsequent image analysis.

In one embodiment, said method comprises IHC (Immunohistochemistry assay), ICC (Immunocytochemistry assay), IF (Immunofluorescence assay) or IHFC (Immunohistofluorescence assay).

In one embodiment, tumor biopsies or FFPE samples are sliced, accordingly treated (e.g., deparaffinized, rehydrated, or subjected to antigen retrieval) and incubated with respective antibodies, that are either labelled or stained with respective labelled antibodies. Detection takes place with suitable imaging methods. Optionally, the slices are counterstained. Image detection and analysis can comprise multispectral image analysis definition of regions of interest, segmentation of tissue into tumor and stroma regions, phenotyping, identification of respective cell types characterized by the respective markers, see e.g. Table 2). The proximity of different cells to one another and/or the density of a given cell type in the sample can be semi-automatically quantified with respective image analysis software (e.g., Inform software, Akoya Bioscience, version 2.4.1). For more details see the experimental description elsewhere herein.

Tumor samples, either fresh, cryopreserved or FFPE, can be used for RNA extraction and gene expression profiling through PCR-based approaches. Among these approaches, RT-qPCR or RNAsequencing or Nanostring can be applied for the assessment of single and/or group of genes expression that can serve as a signature for patient inclusion. High expression of a certain gene or enrichment of a class of gene can constitute a predictive biomarkers. In particular, macrophages related markers can be evaluated.

According to one embodiment, the intratumoral infiltration by macrophages is determined by means of at least one of
detection of CD163 or CD68 expression in the sample, preferably in the tumor tissue identified therein,
detection of CD163⁺ cells or CD68⁺ cells in the tumor, preferably in the tumor tissue identified therein.

In one embodiment, the determination of the intratumoral infiltration by macrophages comprises determination of the density of CD163⁺ cells or CD68⁺ cells in tumor tissue.

In one embodiment, the tumor infiltration is measured as cells/mm² tumor tissue in a tissue slice.

In one embodiment, the tissue slice has a thickness of 7, 6, 5, 4, 3, or 2 μm +/−1 μm. In ne embodiment, the tissue slice has a thickness of 3 μm +/−1 μm.

The inventors noted, surprisingly, that low intratumoral infiltration by macrophages is associated with a high likelihood of improved survival of the patient, and/or better clinical outcome, in response to anti angiogenesis therapy and/or immune checkpoint inhibition therapy, based on the therapeutic agents and regimens discussed herein elsewhere.

In one embodiment, the threshold for determining whether a sample is characterized by “low” intratumoral infiltration by macrophages is a density of macrophages, or, in embodiments, a density of CD163⁺ cells or CD68⁺ cells, of <500, <400, <300, <200, <100, <70, <50 or <30 cells/mm² in the tumor tissue.

In one embodiment, the threshold is <200 cells/mm² in the tumor tissue.

The term “improved survival”, as used herein, refers to a prolonged period of time during which the subject or patient is alive after treatment with a method described herein. Improved survival denotes the greatest probability of staying free of disease progression for an individual suffering from cancer after a particular treatment. It is also used to describe the high percentage of individuals in a group whose disease is likely to remain stable (without showing signs of progression) after a specific period of time, compared to a control group. It is also used to describe the high percentage of individuals in a group whose disease is likely to cure (without showing signs of disease) after a specific period of time, compared to a control group. This parameter can be measured by any of the usual clinical endpoints indicated as “progression-free survival”, “overall survival” and “disease-free survival” used as an indication of the effectiveness of a particular treatment.

The term “better clinical outcome”, as used herein, refers to a condition a patient is in after treatment that is better compared to a patent who was not treated. Such condition can be determined, for example by any suitable diagnosis or scoring method, like for example the Karnofsky Performance Score (KPS), the ECOG/WHO/Zubrod score or the Lansky score. All these approaches are well known to the clinical oncologist.

According to one embodiment, the proximity of lymphocytes to tumor cells is determined by determining the distance between lymphocytes and cells characterized by the presence of a tumor marker.

In one embodiment, the proximity of two or more cell types is measured as mean, average or median distance of the two cell types in μm.

In one embodiment, such tumor marker is a keratin, preferably a cytokeratin, or PSA or CA19-9. Details on these markers, and on the methods how to detect them, are disclosed elsewhere herein.

In one embodiment, the detection of lymphocytes comprises detection of CD8+ cells in the sample. For such purpose, an anti-CD8 antibody can be used, like e.g. the anti-CD8 antibody (ab4055) by Abcam. The skilled person is capable of finding, based on the information provided herein and routine considerations, other suitable antibodies that can be used to detect lymphocytes, in particular CD8⁺ cells.

The inventors noted, surprisingly, that a small distance (=close proximity) of lymphocytes or CD8+ cells to tumor cells is associated with a high likelihood of improved survival of the patient, and/or better clinical outcome, in response to anti angiogenesis therapy and/or immune checkpoint inhibition therapy, based on the therapeutic agents and regimens discussed herein elsewhere.

In one embodiment, the threshold for determining whether a sample is characterized by a small distance (=close proximity) of lymphocytes or CD8+ cells to tumor cells is a mean, average or median distance of <300, <200, <150, <100, <70, <50, or <30 μm.

In one embodiment, the threshold is a mean, average or median distance of <70 μm.

Determination of such mean, average or median distance is preferably carried out in a method comprising an immunoassay. Preferably, such immunoassay method comprises immunostaining and subsequent image analysis.

According to one embodiment, the proximity of macrophages to tumor cells is determined by determining the distance between macrophages and cells characterized by the presence of a tumor marker.

In one embodiment, such tumor marker is a keratin, preferably a cytokeratin. Details on cytokeratin, and on the methods how to detect cytokeratin, are disclosed elsewhere herein.

In one embodiment, the detection of macrophages comprises detection of CD68⁺ cells or CD163⁺ cells in the sample. For such purpose, an anti-CD8 antibody can be used, like e.g. the anti-CD68 antibody clone PG-M1 by Dako or the anti-CD163 antibody clone 10D6 (Leica).

The skilled person is capable of finding, based on the information provided herein and routine considerations, other suitable antibodies that can be used to detect macrophages, in particular CD68⁺ cells or CD163⁺ cells.

The inventors noted, surprisingly, that a large distance (=low proximity) of macrophages, in particular CD68⁺ cells or CD163⁺ cells, to tumor cells is associated with a high likelihood of improved survival of the patient, and/or better clinical outcome, in response to anti angiogenesis therapy and/or immune checkpoint inhibition therapy, based on the therapeutic agents and regimens discussed herein elsewhere.

In one embodiment, the threshold for determining whether a sample is characterized by a large distance (=low proximity) of Macrophages or CD163+ or CD68+ cells to tumor cells is a mean, average or median distance of ≥30, ≥50, ≥70, ≥100, ≥150, ≥200 or ≥300 μm

In one embodiment, the threshold is a mean, average or median distance of ≥50 μm.

As set forth above already, the terms small distance and close proximity are used interchangeably. As set forth above already, the terms large distance and low proximity are used interchangeably.

Determination of such mean, average or median distance is preferably carried out in a method comprising an immunoassay. Preferably, such immunoassay method comprises immunostaining and subsequent image analysis.

According to one embodiment of the method, determination of the presence or absence of one or more nearby tertiary lymphoid structures comprises at least one of
detecting the expression, presence or absence of CD3 and CD20 in the sample
staining the sample with haematoxyin and Eosin (H&E)
detecting the presence or absence of DC-Lamp⁺ mature dendritic cells (mDCs) and/or CDS⁺ T cells, and/or
detecting the expression, presence or absence of at least one of CD21 (also known as CR2), CD35 (also known as CR1) and CD23 (also known as FcεRII)
detecting the expression, presence or absence of at least one of CD20 and CD19, and/or
detecting the expression, presence or absence of at least one of PNAd

As used herein, the term DC-Lamp+ mature dendritic cells relates to dendritic cells (DC) which express LAMP3, also known as DC-LAMP (Dendritic cell lysosomal associated membrane glycoprotein).

As used herein, the term PNAd relates to Peripheral node addressin (PNAd), which is a sulfated and fucosylated glycoprotein recognized by the prototypic monoclonal antibody, MECA-79. PNAd marks high endothelial venules (HEV), which are crucial for the recruitment of lymphocytes into lymphoid tissue

The inventors noted, surprisingly, that the presence of one or more nearby tertiary lymphoid structures is associated with a high likelihood of improved survival of the patient, and/or better clinical outcome, in response to anti angiogenesis therapy and/or immune checkpoint inhibition therapy, based on the therapeutic agents and regimens discussed herein elsewhere.

In one embodiment, the expression, presence or absence of CD20 on B cell follicles and the expression, presence or absence of CD3 in an adjacent T cell zone is detected simultaneously.

These methods, and others, are disclosed in Sates Friedman et al (2019), the content of which is incorporated herein by reference for enablement purposes.

The following table summarizes the different features provided in the method according to the invention and their meaning for survival or clinical outcome:

| | | association with improved | |
|---|---|---|---|
| | | survival/better clinical | |
| | | outcome, in response to | |
| | | anti angiogenesis therapy | |
| | | and/or immune checkpoint | |
| feature | Example marker | inhibition therapy | treshold |
| intratumoral | CD163 orCD68 | low intratumoral infiltration | density of CD163⁺ cells or CD68⁺ cells, |
| infiltration by | vs PanKer or | | of ≤500, ≤400, ≤300, ≤200, ≤100, ≤70, ≤50 |
| macrophages | selected CytoKer | | or ≤30 cells/mm² in the tumor tissue. |
| | (CK) | | |
| proximity of | CD8 vs PanKer or | small distance (=close | mean/average/median distance |
| lymphocytes | selected CytoKer | proximity) of lymphocytes | of ≤300, ≤200, ≤150, ≤100, ≤70, ≤50, |
| to tumor cells | (CK) | or CD8+ cells to tumor cells | or ≤30 μm. |
| proximity of | CD163 or CD68 vs | large distance (=low | mean/average/distance |
| macrophages | PanKer or selected | proximity) of macrophage or | of ≥30, ≥50, ≥70, ≥100, ≥150, ≥200 |
| to tumor cells | CytoKer (CK) | CD163+ or CD68+ cells | or ≥300 μm |
| | | to tumor cells | |
| presence or | many (see text) | Presence of TLS nearby to | qualitative feature |
| absence of one | | tumor | |
| or more nearby | | | |
| tertiary lymphoid | | | |
| structures (TLS) | | | |

According to one embodiment, the method further comprises the step of determining, in the same sample the maturity status of the TLS. According to one embodiment, determination of the maturity status of the TLS comprises detecting, on a protein- or mRNA basis, the expression, presence or absence of CD23 (also known as FcεRII). According to one embodiment, determination of the maturity status of the TLS comprises determining the density of CD23 positive TLS. The dynamics of TLS maturation have so far remained unclear. Generally, three sequential TLS maturation stages can be described and are characterized by increasing prevalence of follicular dendritic cells and mature B-cells:

[1] Early TLS, composed of dense lymphocytic aggregates without FDCs,
[2] Primary follicle-like TLS, having FDCs but no GC reaction, and
[3] Secondary follicle-like TLS, having an active GC reaction.

According to one embodiment of the method,
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
   is indicative for
a high likelihood of improved survival of the patient, and/or
better clinical outcome
   in response to anti-angiogenesis therapy and/or immune checkpoint inhibition therapy, based on the therapeutic agents and regimens discussed herein elsewhere.

According to another aspect of the invention, a kit for carrying out a method according to the above description is provided, said kit comprising means for detecting at least one of a) intratumoral infiltration by macrophages, and/or
b) proximity of lymphocytes to tumor cells, and/or
c) proximity of macrophages to tumor cells, and/or
d) presence or absence of one or more nearby tertiary lymphoid structures (TLS)

According to one embodiment, said kit comprises at least one oligonucleotide comprising a nucleotide sequence which is capable of hybridizing to a nucleic acid encoding for at least one of

### CD163,

### CD68,

which oligonucleotide is selected from the group consisting of
an amplification primer
a labelled probe, and/or
a substrate bound probe
   wherein said hybridization occurs under stringent conditions, namely conditions under which a probe or primer will hybridize to its target subsequence, but to no other sequences.

Said nucleic acid is preferably an mRNA sequence, as shown in the following table.

| Target | Genomic sequence | mRNA sequences |
|---|---|---|
| CD163 | NG_029826.1 | NM_001370145.1 (isoform b) |
| | RefSeqGene | NM_001370146.1 (isoform c) |
| | | NM_004244.5 (isoform a precursor) |
| | | NM_203416.3 (isoform b precursor) |
| CD68 | | NM_001040059.2 (isoform B precursor) |
| | | NM_001251.3 (isoform A precursor) |

Further, said kit may optionally comprise at least one oligonucleotide comprising a nucleotide sequence which is capable of hybridizing to a nucleic acid encoding for at least one of CD8; Keratin; one or more Cytokeratins; CD3; CD20; DC-LAMP; CD21; CD35; CD23; CD20; CD19, and or PNAd. To further stratify the tumor in greater detail, said kit may optionally comprise at least one oligonucleotide comprising a nucleotide sequence which is capable of hybridizing to a nucleic acid encoding for at least one of cMAF, PD1, PD-L1 or IDO1.

In one embodiment, such primer or probe comprises a nucleic acid sequence that comprises a substretch of at least one of the mRNA sequence set forth above, or a substretch of its reverse complement.

Such primer or probe may have a length of between 10 and 40 bases, preferably between 18 and 30.

Based on this information, the skilled person is able to design suitable primers and probes, based on his routine experience and suitable literature and software tools, like e.g. the software “Primer BLAST” offered by the US National Library of Medicine.

According to one embodiment, the kit is suitable for at least one of
Fluorescent in situ hybridization (FISH),
In situ PCR,
Real time PCR, and/or
Reverse transcription PCR/qPCR

According to one other embodiment, the kit comprises at least one immunoligand capable of binding to at least one of CD163; CD68; CD8; Keratin; one or more Cytokeratins; CD3; CD20; DC-LAMP; CD21; CD35; CD23; CD20; CD19, and or PNAd in an immunoassay

Further markers can also be used in the context of the invention to stratify the tumor in greater detail, including cMAF, PD1, PD-L1, IDO1.

In one embodiment, at least one of said immunoligands is an antibody.

According to one embodiment, said immunoassay is at least one selected from the group consisting of
IHC (Immunohistochemistry assay)
ICC (Immunocytochemistry assay) and/or
IF (Immunofluorescence assay)

Preferably, such method comprises immunostaining and subsequent image analysis.

According to another aspect of the invention, an anti-angiogenic drug (in the manufacture of a medicament) is provided for use in the treatment of a patient that suffers from or is at risk of developing a solid tumor, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)

According to another aspect of the invention, a combination of an anti-angiogenic drug and an immune checkpoint inhibitor (in the manufacture of separate coadministrable medicaments) is provided for use in the treatment of a patient that suffers from or is at risk of developing a solid tumor, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
   wherein the combination of the two drugs is administered to a patient concomitantly or consecutively.

As used herein the term “concomitantly” means that the two drugs are administered to the patient at the same time, yet in the same or in different dosage units.

As used herein the term “consecutively” means that the two drugs are administered to the patient at different times, yet to obtain a synergistic or complimentary effect.

According to another aspect of the invention, a method of treating a patient that suffers from or is at risk of developing a solid tumor with an anti-angiogenic drug is provided, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
   wherein said method comprises administration of the drug in at least one therapeutically effective dosis.

According to another aspect of the invention, a method of treating a patient that suffers from or is at risk of developing a solid tumor with a combination comprising an anti-angiogenic drug and an immune checkpoint inhibitor is provided, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
   wherein said method comprises administration of the combination of the two drugs in at least one therapeutically effective dosis, and wherein the combination of the two drugs is administered to a patient concomitantly or consecutively.

With regard to the above embodiments, to avoid lengthy repetitions, the same considerations apply as disclosed elsewhere herein regarding
a) methods and thresholds for determining intratumoral infiltration by macrophages, proximity of lymphocytes to tumor cells, proximity of macrophages to tumor cells, and/or presence of one or more nearby tertiary lymphoid structures (TLS)
a) the nature of the anti-angiogenic drug and the immune checkpoint inhibitor, and preferred combinations thereof, and
b) the types of tumor.

In one embodiment, the anti-angiogenic drug is a tyrosin kinase inhibitor, preferably Regorafenib. In one embodiment, the immune checkpoint inhibitor is an anti PD-L1 antibody, preferably Avelumab.

The combination of Regorafenib and Avelumab is particularly preferred.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus.

### Methods

Study Design and Participants

REGOMUNE is a single-arm, open-label, multicentre phase II basket study for which patients were recruited from 4 French sites. In the colorectal cancer cohort, patients were eligible if they were aged at least 18 years and had histologically proven MSS advanced or metastatic colorectal cancer (CRC); Eastern Cooperative Oncology Group performance status of 0-1; Measurable disease according to RECIST; at least 1 previous line of systemic treatment; adequate hematological, renal, metabolic and hepatic functions.

Blood test included assessment of blood cell count, alanine aminotransferase, aspartate aminotransferase, alkaline phosphatase, albumin, bilirubin, lipase, creatinine phosphokinase, coagulation test, creatinine, and urea nitrogen. Key exclusion criteria included previous treatment with Avelumab or Regorafenib, previous treatment anti-PD-1, anti-PD-L1, anti-PD-L2, anti-CD137, or anti-Cytotoxic T-lymphocyte-associated antigen-4 (CTLA-4) antibody, active auto immune disease, arterial or venous thrombotic or embolic events such as cerebrovascular accident, deep vein thrombosis or pulmonary embolism within 6 months before the study, known active hepatitis B or C, persistent proteinuria>3.5 g/24 hours, major surgical procedure within 28 days before the study, non-healing wound, non-healing ulcer, or non-healing bone fracture, any bleeding event≥CTCAE Grade 3 within 4 weeks prior to the start of the study, uncontrolled hypertension despite optimal medical management, congestive heart failure≥New York Heart Association class 2, [symbol] unstable or new-onset angina, myocardial infarction less than 6 months before the study, uncontrolled cardiac arrhythmias. As required by French regulations, the protocol was approved by a central institutional review board that reviewed the appropriateness of the clinical trial protocol as well as the risks and benefits to study participants. All patients provided written, informed consent.

### Procedures

After an assessment of eligibility, patients received Regorafenib 160 mg per day on a three weeks on/one week off schedule, in cycles of 28 days. Avelumab treatment began on Day 15 cycle 1, by intravenous infusion once every 2 weeks. Treatment was continued until disease progression, unacceptable toxicity, investigator's decision to discontinue, or withdrawal of patient consent. Participants were monitored for adverse events at every follow-up assessment. Adverse events were graded according to National Cancer Institute Common Terminology Criteria for Adverse Events version 5.0. Laboratory assessments were done at baseline, and every 2 weeks afterwards until treatment discontinuation. Regorafenib dose modifications to manage adverse events were allowed (see study protocol). The dose of Regorafenib could be reduced to 120 mg and then to 80 mg. Dose interruptions were allowed based on the clinical situation. Patients requiring a delay of >4 weeks since the last dose of Regorafenib had to permanently discontinue Regorafenib but could continue Avelumab if this were considered as appropriate. No dose reduction of Avelumab was allowed. Dose interruptions were allowed based on the severity of immune related adverse events. Patient requiring two or more consecutive cancellation of Avelumab injection had to permanently discontinue Avelumab and were allowed to continue Regorafenib. Tumor lesions were assessed according to RECIST version 1.1 at baseline (within 4 weeks prior to Cycle 1 Day 1), and every 8 weeks until disease progression or the start of another treatment. Tumor samples were collected at baseline and cycle 2 day 1 for all consenting patients in order to assess the impact of treatment on tumor microenvironment and to identify potential biomarkers associated with outcome.

### Outcomes

The primary endpoint was the 6-months objective response rate under treatment defined as the proportion of patients with objective response (confirmed or unconfirmed) under treatment based on adapted RECIST 1.1 after centralized radiological review.

Secondary objectives included best overall response, objective response rate at 6 months, 6-months progression-free rate, 1-year progression-free survival, 1-year overall survival and safety. Best overall response was defined as the best response across all time points. Progression-free survival was defined as the time from study treatment initiation to the first occurrence of disease progression or death from any cause. Overall Survival is defined as the time from study treatment initiation to death from any cause.

Statistical Analysis

The study was based on a Bayesian approach following an adaptive trial design, with objective response as the primary endpoint. A maximum sample size of 50 patients was planned. The analysis of the primary endpoint was carried out sequentially, with [symbol] interim analyses planned after 16-week follow-up of the first 10 patients and then every 5 patients. Inclusions were not suspended between interim analyses. The probability of success was estimated from a beta-binomial model. Maximal response probability threshold and minimal response probability threshold have been defined as follows: 5% versus 20%. At each interim analysis, a stopping rule for inefficacy recommended stopping the trial if there was a high predictive probability (≥80%) that the ORR was lower or equal to the minimal response probability threshold.

All enrolled patients who initiated the study treatment were included in the safety analysis. The efficacy population included all participants who met the eligibility criteria and who received at least one complete or two incomplete treatment cycles. The median follow-up was calculated using the reverse Kaplan-Meier method. Survival endpoints (progression-free survival and overall survival) were described using the Kaplan-Meier method. Data for patients who were alive and progression-free were censored at the date of the last follow-up. Quantitative variables were described using the median and range, and qualitative variables were described using frequency, percentage and 95% confidence interval (binomial law). All eligible and assessable patients for efficacy were included in the denominator for the calculation of the proportions. Estimated parameters were reported with two-sided 95% CIs. p values less than 0.05 (typically <0.05) were statistically significant. Statistical analyses were done using SAS software (version 9.4). This study was registered with ClinicalTrials.gov, number NCT03475953.

Tissue Sample Analysis

Tumor biopsies were collected at baseline and at Day 1 of cycle 2. These samples were analyzed to characterize the impact of Regorafenib combined with Avelumab on tumor microenvironment and to identify potential predictive biomarkers of clinical benefit. Immuno-histofluorescence analysis was performed on the automated Ventana Discovery XT staining platform (Ventana Medical Systems). Slides of tumor tissue were deparaffinized in xylene and hydrated in serial alcohol solutions. Antigen retrieval was performed by heat-induced epitope retrieval method using standard CC1 (tris based buffer) pH8 (Ventana Medical Systems). The slides were incubated with the following primary antibodies: anti-CD8 (clone C8/144B, Dako, dilution 1/25), anti-CD163 (clone 10D6, Leica, dilution 1/100eme), anti-PDL1 (clone QR1, Diagomics, dilution 1/100), (optionally also anti-IDO1 (clone UMAB126, Origene, dilution 1/200), anti-PD1 (clone NAT105, Roche, ready-to-use) anti-Pan Keratin (clone AE1/AE3/PCK26, Roche, ready-to-use)). Bound primary antibodies were detected using either OmniMap anti-Ms or Rb-HRP with Opal detection kit (Akoya Bioscience). The slides were counterstained with spectral DAPI (Perkin Elmer) and cover-slipped. Stained slides were imaged on the multispectral slide analysis system (Vectra Polans, Perkin Elmer) and analyzed using Inform image analysis software (Perkin Elmer, version 2.4.1). Briefly, multispectral images obtained were unmixed using spectral libraries that were previously built from images stained for each fluorophore (monoplex), using the inForm Advanced Image Analysis software (inForm 2.4.1, Akoya Bioscience). For each case, regions of interest in the whole biopsy were defined by manual annotation by a pathologist. Machine learning was then applied in order to i) segment tissue into tumor and stroma regions—taking into account cell morphology, density, and the different stainings (Pan Keratin, CD8, CD163, PDL1, IDO1, PD1) ii) segment each cells with definition of nucleus, cytoplasm and membrane compartments. After these segmentation steps, phenotyping was applied in order to define cells positive for the following markers: Pan Keratin, CD8, CD163 and PD1. Fluorescence intensity (Mean Fluorescence intensity) was calculated for both PDL1 and IDO1 in each cell. Density of the following immune cells—CD163+ and CD8+—was semi-automatically quantified with the Inform software (Akoya Bioscience, version 2.4.1) and was defined as the number of CD163+ or CD8+ cells per mm² of tumor surface. At the same time distance of each CD8+ T cells from a tumor cell (defined as Pan Keratin positive) was calculated, averaged for each case and was expressed in μm.

### Results

Between Nov. 26, 2018 and Oct. 4, 2019, 48 patients were recruited to the study. 46 patients were eligible, 43 were eligible and assessable for the efficacy endpoint. Three patients were not eligible for the efficacy assessment due to protocol deviations (FIG. 1). Characteristics of the patients are summarised in table 1. The median age was 62 years (range 26-83), and 35% of the patients were women. Seventy-eight percent of the patients had already received systemic treatment for advanced disease, with a median of 3 (range 0-7) previous lines.

In the efficacy population and after a median follow-up of 7.2 months (95% CI 6.4-8.1), 29 (67.4%) were still alive, with 12 (27.9%) still under treatment.

Among the 43 patients who were eligible and assessable for efficacy, 3 were not evaluable for RECIST response: two patients discontinued study because of clinical progression before the planned tumor evaluation, one patient discontinued study based on investigator decision before planned tumor evaluation (FIG. 1).

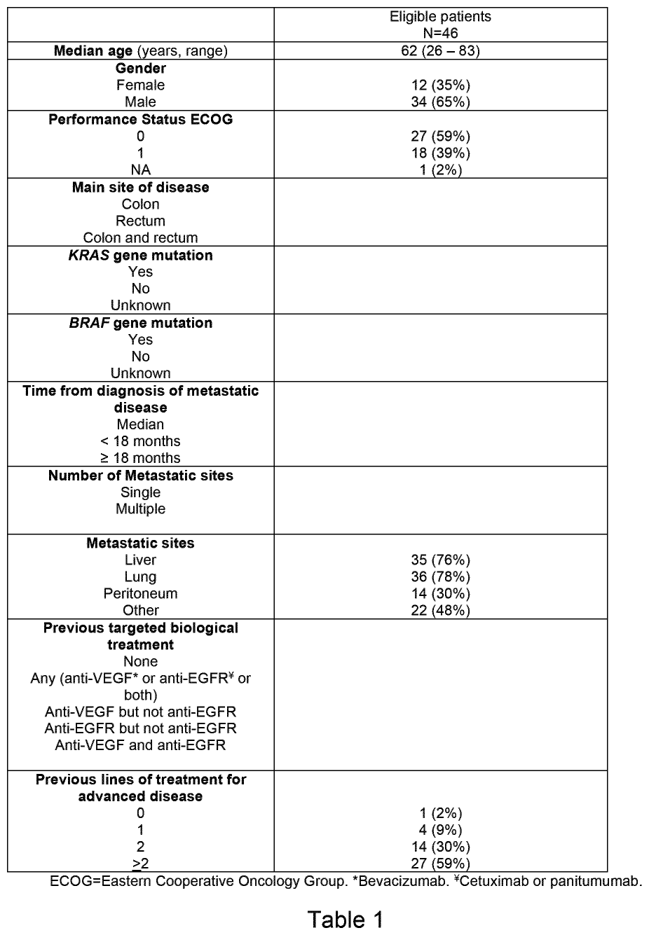

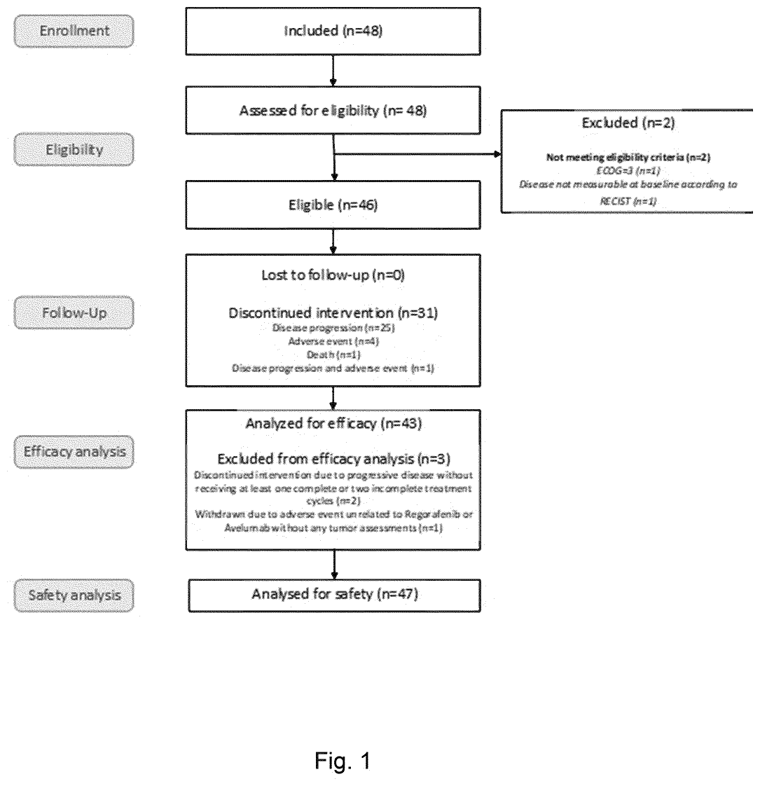

No patient achieved an objective response and as such, primary efficacy criterion was not reached. Regarding the best overall response, 23 (53.5%) showed stable disease, including 12 (28%) with tumor shrinkage (range from -% to -%) (FIG. 2). Seventeen patients (39.5%) had progressive disease.

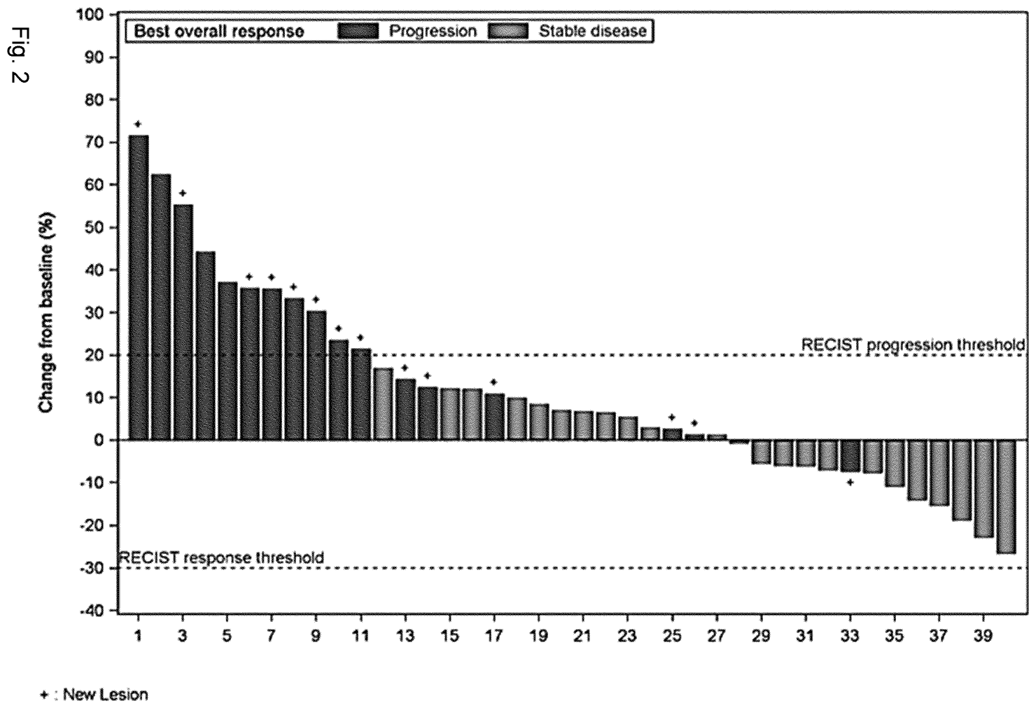

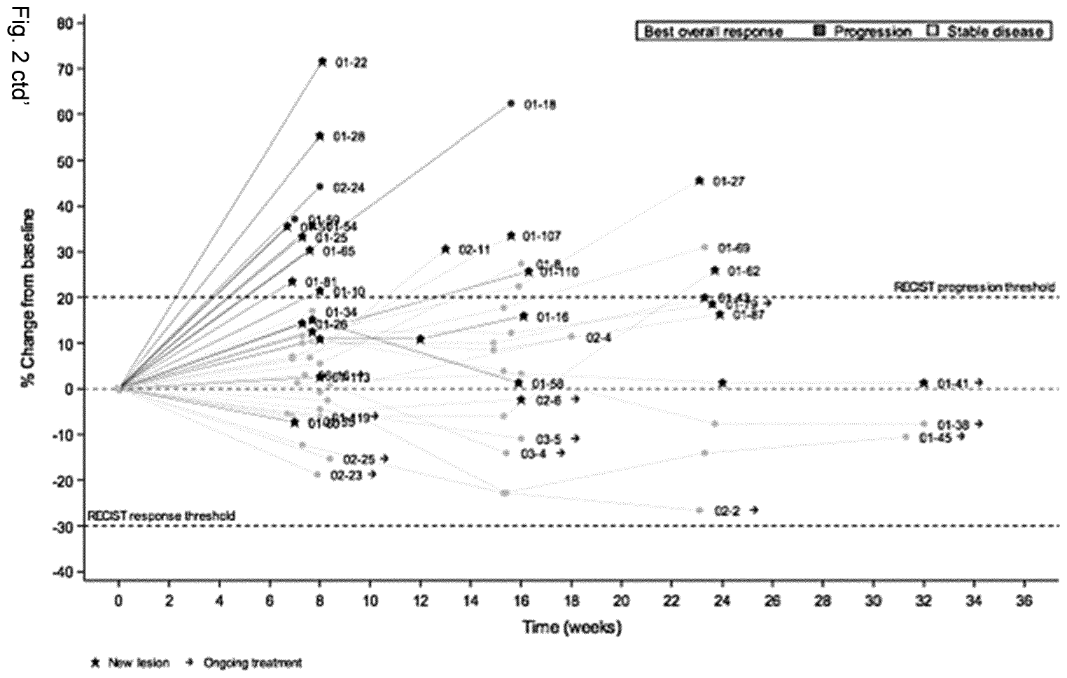

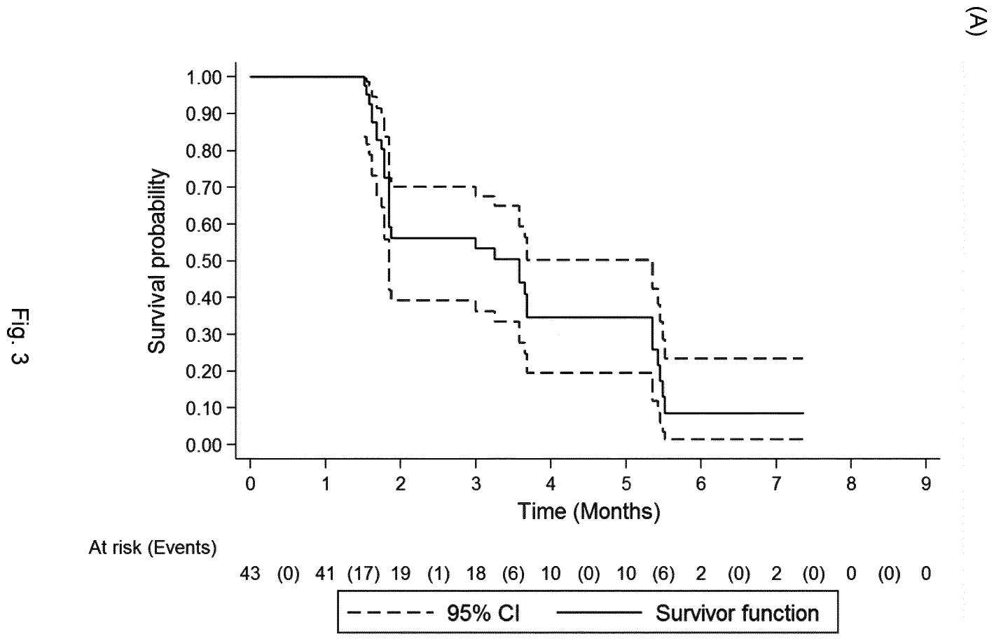

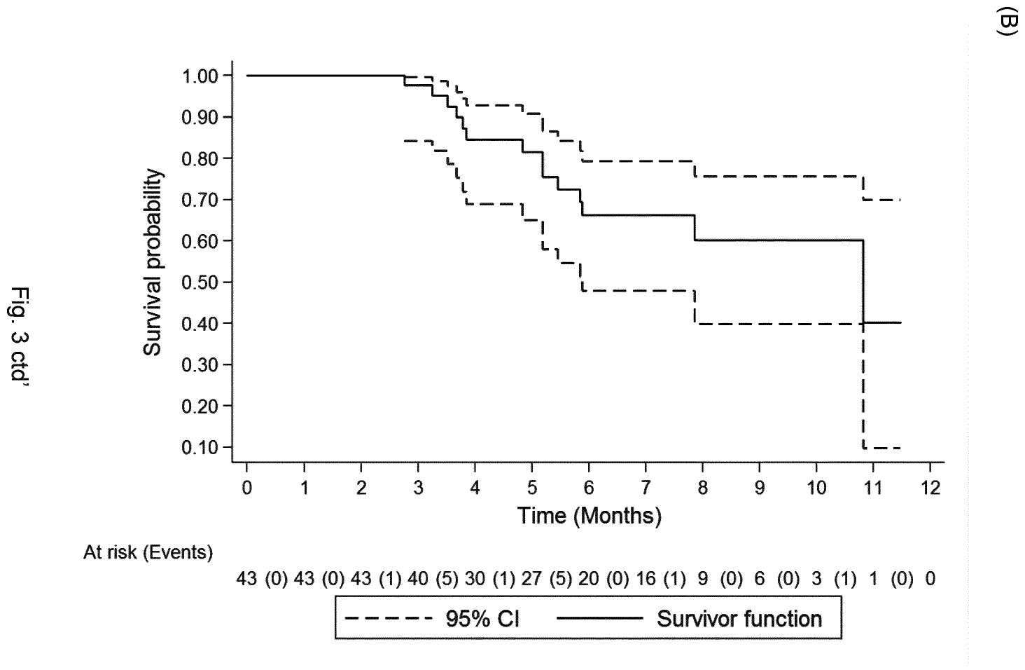

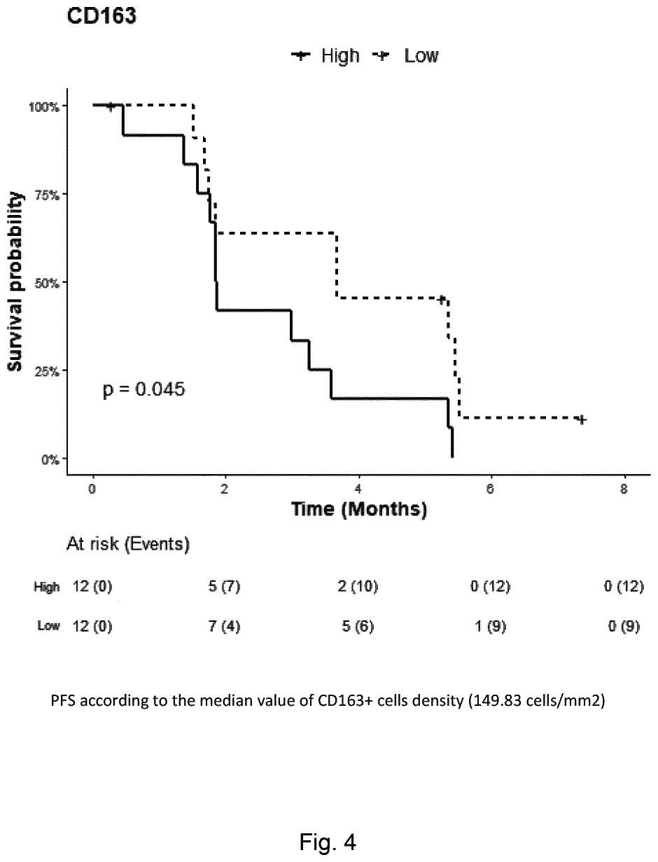

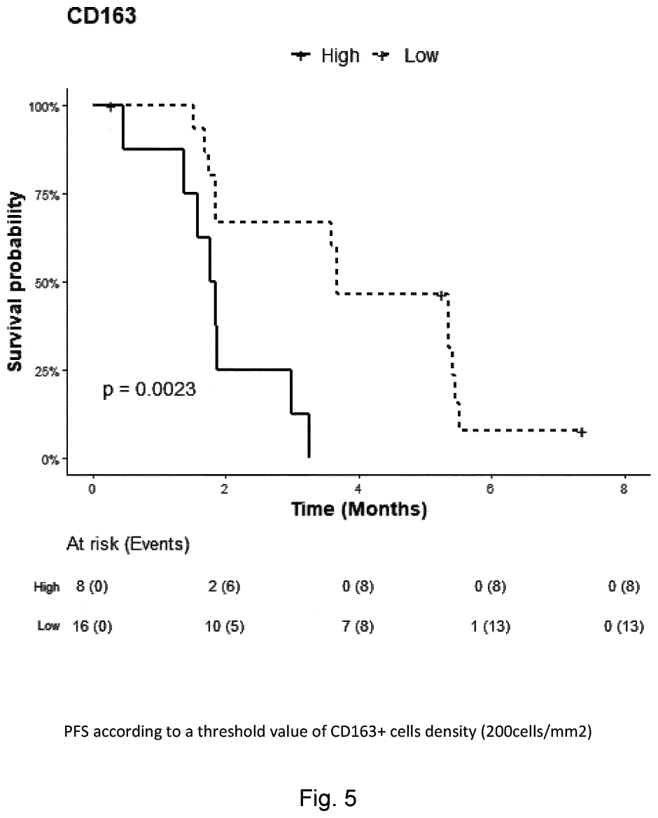

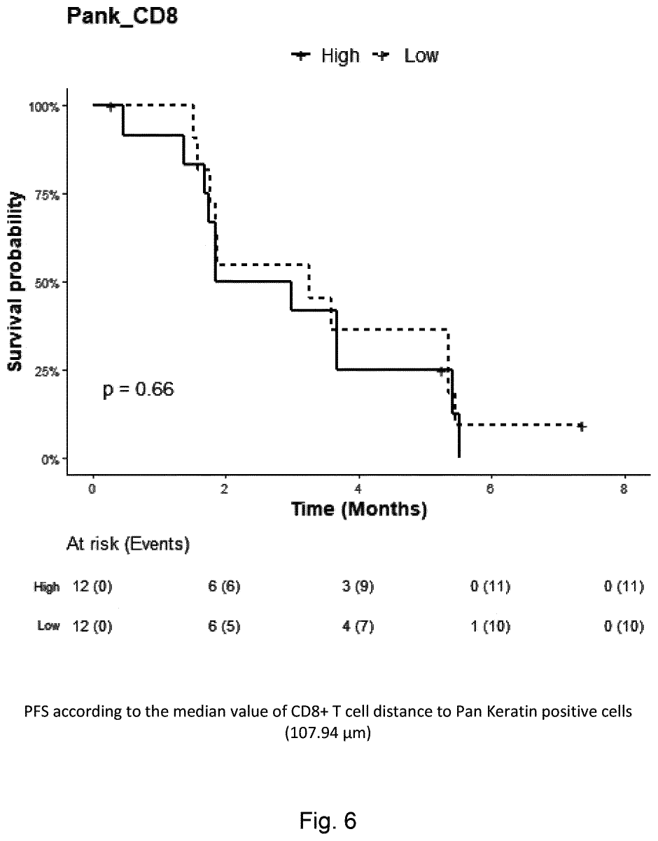

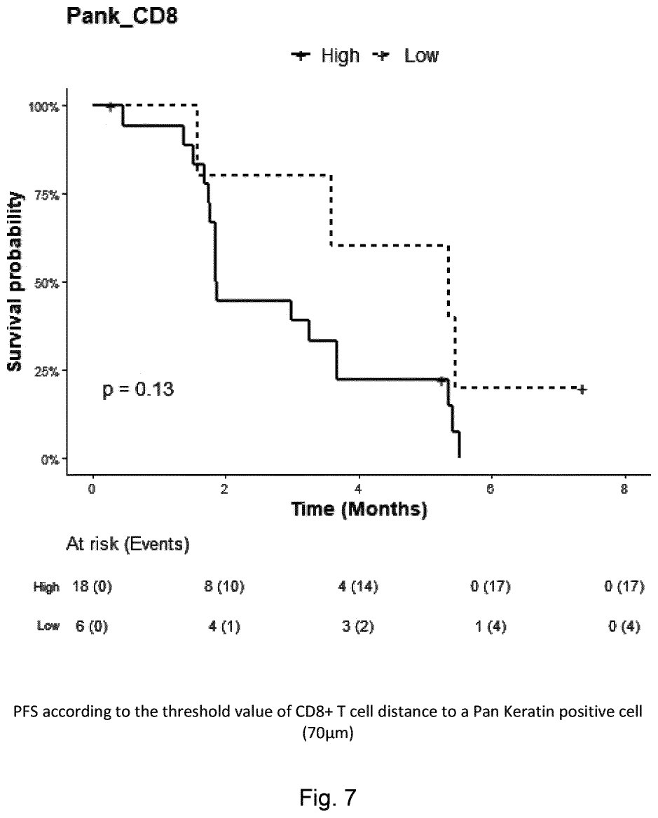

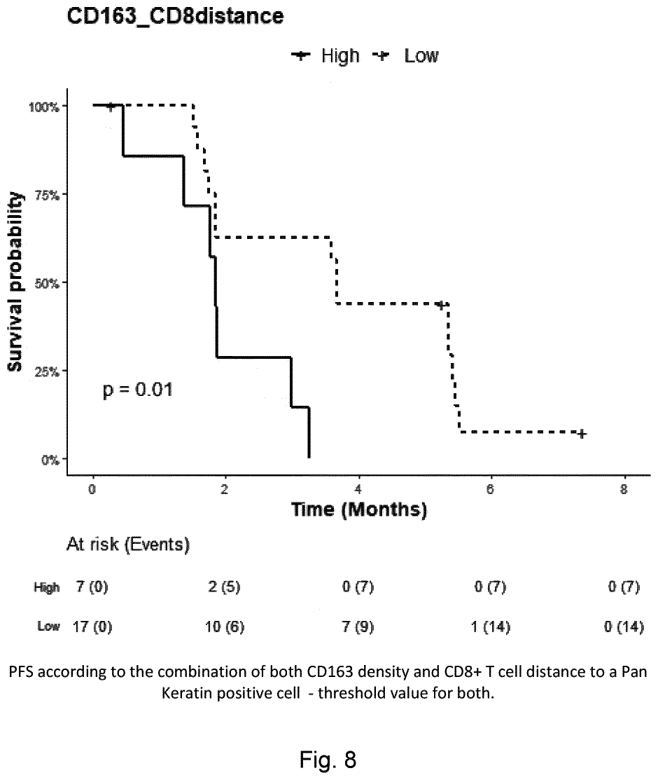

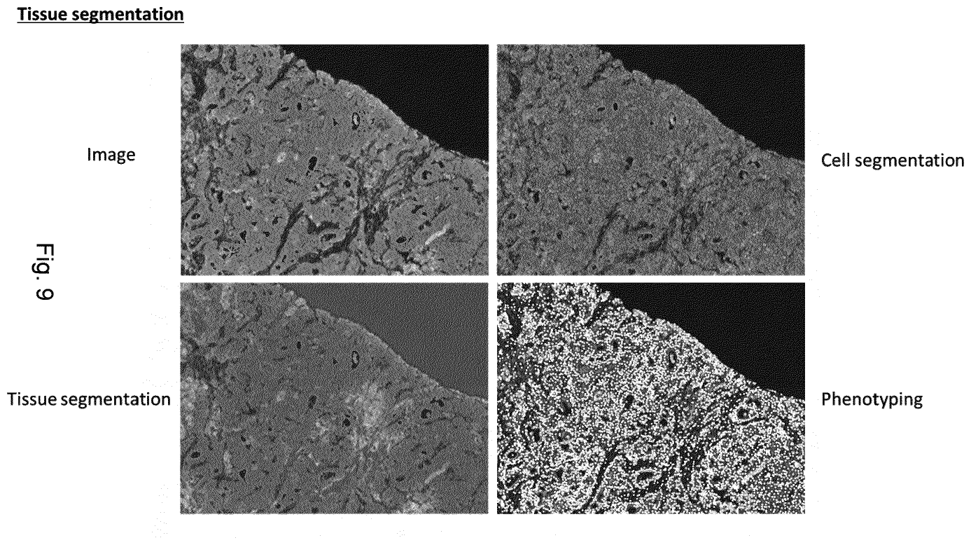

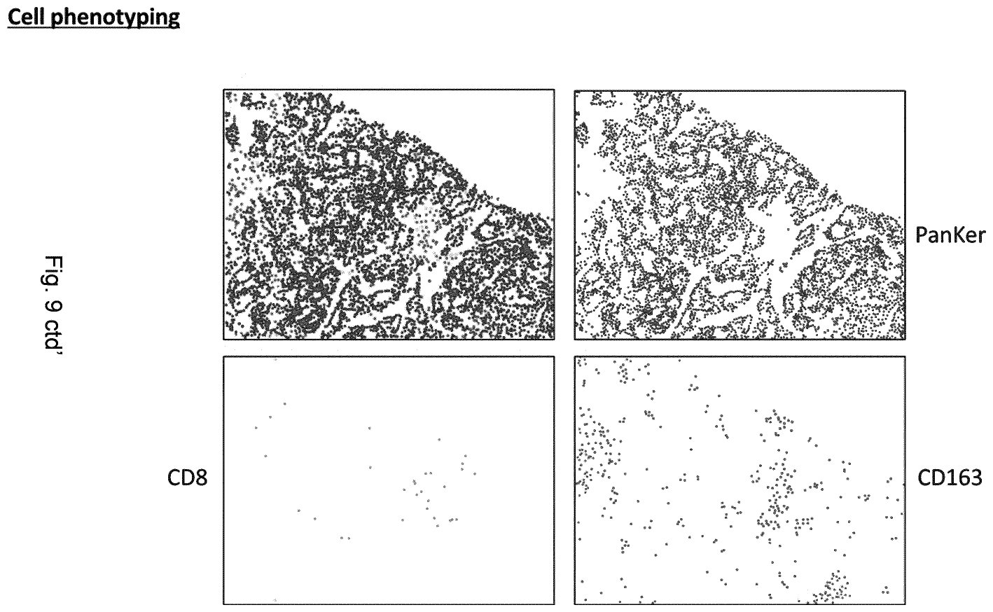

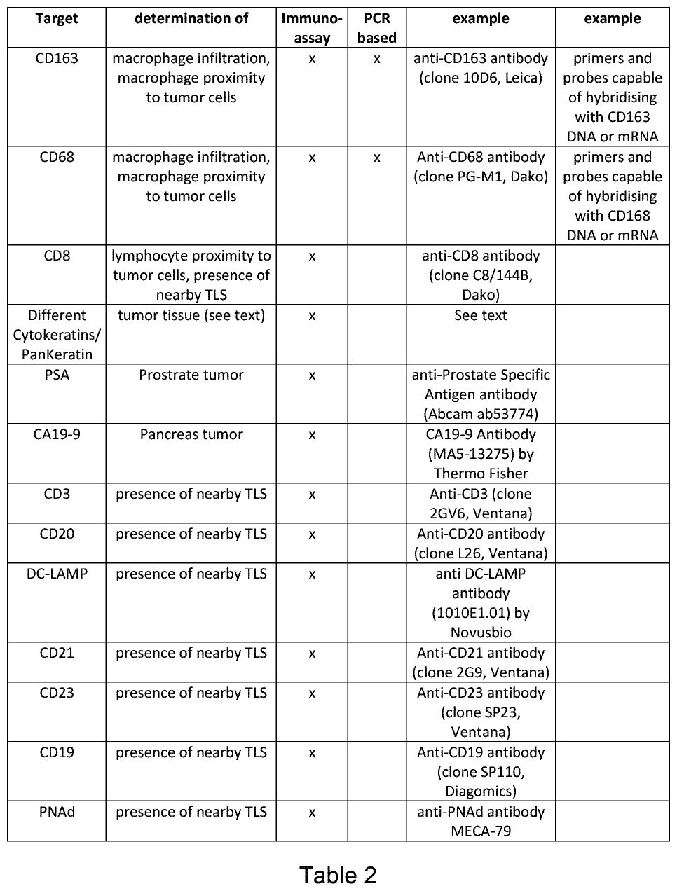

Median progression-free survival and overall survival were 3.6 months (95% CI 1.8-5.4) and 10.8 months (95% CI 5.9—NA) (FIG. 3) respectively.

Forty-seven patients received at least one dose of Regorafenib and/or Avelumab and were therefore evaluated for safety. Treatment was generally well tolerated, although virtually every patient had grade 1 or 2 adverse events (AE) related to therapy. The most common clinical treatment-related AE were anorexia, diarrhea, palmar-plantar erythrodysesthesia syndrom, fatigue, hypertension, mucositis, and myalgia. As expected, the most common-treatment related lab abnormality were transaminitis and TSH increase. At least one serious adverse event was reported in 22 patients (46.8%). 41 patients experienced dose reduction or temporary treatment discontinuation because of a drug-related adverse event (41 patients for Regorafenib and 17 patients for avelumab). No patient died from drug-related toxicity.

Overall, baseline tumor samples and paired biopsies (baseline and cycle 2 day 1 biopsies) were available for 24 and 15 patients, respectively. By analysing paired tumor biopsies, we observed a significant increase in T CD8 cell infiltration, and a significant decrease in tumor-infiltrating M2 macrophages, respectively. Patients with increased infiltration by CD8+ T cell at cycle 2 Day 1 compared to baseline had significantly better progression-free survival and overall survival than patients with no change.

PDL1 was expressed by some tumor cells and immune cells. PDL1 status was not correlated with PFS neither with OS. In order to identify potential predictive biomarkers of efficacy, we quantified the different immune cell subsets as well as their spatial context on pre-treatment samples and correlated them with PFS and OS. Interestingly, we found that high level tumor-infiltrating M2 macrophages at baseline was significantly associated with adverse outcome (progression-free survival: 1.9 vs 3.7 months, overall survival: 4.8 months versus not reached). Moreover, patients bearing tumors with the shortest distance between CD8 T lymphocytes and cancer cells had a significantly better outcome than patients with a longer distance. Combining low tumor-associated macrophages infiltration and low distance between tumor cells and CD8+ T cell enabled the identification of a subgroup of patients (6/24, 25%) more likely to benefit from Regorafenib+Avelumab combination: median progression-free survival of 5.3 vs 1.9 months; median overall survival was not reached vs 5.3 months.

### DISCUSSION

Dozens of clinical trials are currently ongoing in order to investigate the potential therapeutic role of anti-angiogenic therapy combined with immune checkpoint inhibitors in solid tumors. Such combinations have already been assessed with success in renal cell carcinomas as illustrated by the recent approval of pembrolizumab plus axitinib and Avelumab plus axitinib in the first-line setting for patients with advanced disease.

This is the first report of a clinical study investigation the combination of antiangiogenic agent with anti-PDL1 in patients with MSS-colorectal cancer. Thanks to our comprehensive analysis of tumor samples, we showed that this combination has a significant impact on the tumor microenvironment of colorectal cancer patients. We observed a significant reduction in macrophage infiltration on treatment. Regorafenib was shown to reduce immunosuppressive macrophage infiltration in a preclinical model of metastatic colorectal cancer. Our results confirmed this effect in the clinical setting. Several studies have shown that tumor associated macrophages play a crucial role in CRC tumorigenesis by promoting angiogenesis and metastasis due to its ability to secrete VEGF. Interestingly, in our study, patients with the highest level of macrophage infiltration at baseline had the worst outcome suggesting the potential role of this biomarker in the selection of patients who are more likely to benefit from this approach.

We observed also a significant increase in T CD8 infiltration and this change was associated with improved outcome. interestingly, a recent assessment of pre- to post-treatment changes in 20 localized MSS colorectal cancer patients treated with immune-checkpoint inhibitors in the neoadjuvant setting also showed significant increases in CD8⁺ T cell which was more pronounced in patients with good pathological response |16]. Our results suggest that immune checkpoint inhibition leads to immune activation also in metastatic MSS colorectal, even when this is not associated with radiological response as per RECIST.

PD-L1 expression is considered as an important biomarker to guide treatment with immune checkpoint inhibitors in several solid tumors such as non-small cell lung cancer, head and neck or gastric cancer. In our study, PDL1 status was not correlated with PFS neither with OS. This result is in line with those of the neoadjuvant study mentioned above. In that study PD-L1⁺ expression was not correlated with the rate of pathological response. Several studies have shown that the proportion PD-L1⁺ cases is significantly higher in dMMR/MSI-H CRC than in pMMR/MSS CRC. However, even in, dMMR PDL1 expression is not clearly associated with clinical outcome. Altogether, these data indicate that PD-L1 expression status may not represent a reliable biomarker in CRC to guide immune checkpoint therapy therapy as this is the case for as some tumor types such as melanomas. We, therefore, focused our study on the tumor immune microenvironment by analysing baseline and paired tumor samples obtained before treatment at cycle 2 Day 1 respectively. Indeed, computational pathology has the potential to improve patient stratification thanks to a better understanding of tumor-host interaction. By using advanced multiparametric imaging applications, we were able to identify a group of patients characterized by low macrophage infiltration and low distance between CD8 T cells and tumor cells, who are more likely to benefit from the Regorafenib-Avelumab combination. Further studies are needed to confirm prospectively the predictive value of these biomarkers.

With a median PFS and a median OS of 3.6 and 10.8 months, respectively the results of our study compared favourably with the median PFS of 1.9 months and of OS of 6.4 months observed with Regorafenib used as a single agent in the pivotal study which lead to approval. However, we did not observe the same range of activity than in the REGONIVO the results of which were presented in abstract form. This study which investigated the combination of Regorafenib and nivolumab in digestive tumors enrolled 24 Japanese patients with metastatic MSS colorectal cancer. The objective response rate was 33% and the median PFS was 6.3 months. Several explanations can explain these differences of outcome with the results of our study.

Most of the patients required dose reductions of Regorafenib due to adverse events. However, this proportion was not different to that observed in clinical studies investigating Regorafenib as single agent in colorectal cancer. In the CORRECT (as well as in the CONCUR study which included Asian patients), up to 54% of patients had grade 3 or 4 treatment-related adverse events that needed some dose reduction. The safety profile observed in our study did not show any signal in favour of an increase of toxicity of Regorafenib or Avelumab in comparison with their use as single agent. The optimal dose of anti-angiogenic to combine with immune checkpoint inhibition remain a question of debate. Indeed, overcoming immunosuppression induced by VEGF as well as by its blockade may be potentially achieved by using different strategies. One approach could be careful titration of VEGF inhibition to inhibit VEGF pathway and angiogenesis while avoiding excessive pruning and hypoxia. For example, Huang et al. showed that dose-titrated anti-VEGFR2 antibody therapy can alleviate hypoxia (via vascular normalization) and potentiate the effects of vaccination in a mouse model of breast cancer. By using a low-dose anti-VEGFR2 antibody, effector T-cell infiltration increased compared with high-dose anti-VEGFR2 treatment. In addition, tumor-infiltrating macrophages showed a more immune stimulatory (M1) phenotype. Similar results have reported using TNF-α blockade in the mouse models. While TNF-α blockade itself can alleviate immune suppressive phenotype on immune cells, vascular destruction by TNF-α blockade resulted in increased tumor hypoxia and paradoxically caused immune suppression. Interestingly, a large fraction of the patients with tumor shrinkage and of patients which showed an increase in lymphocytic infiltration on sequential tumor biopsies experienced dose reduction suggesting that high dose are not indispensable to induce immune activation and synergistic clinical activity.

The main limitation of this study is its non-randomized design. Indeed, by minimizing many sources of potential bias, randomized, controlled clinical trials provide the most robust information about the effects of investigational drugs. However, this first proof of concept study provides robust information that pave the way fir successful immunotherapy strategies for a devastating disease that cause more than one million deaths each year worldwide.

### REFERENCES

The disclosures of these documents are herein incorporated by reference in their entireties.
Sautes-Fridman, C., Petitprez, F., Calderaro, J. et al. Tertiary lymphoid structures in the era of cancer immunotherapy. Nat Rev Cancer 19, 307-325 (2019).

What is claimed is:

1. An in vitro method for the prognosis of disease progression in a patient that suffers from or is at risk of developing a solid tumor, said method comprising determining, in one or more samples from said patient, at least one of
a) intratumoral infiltration by macrophages, and/or
b) proximity of lymphocytes to tumor cells, and/or
c) proximity of macrophages to tumor cells, and/or
d) presence or absence of one or more nearby tertiary lymphoid structures (TLS)
wherein the prognosis is an assessment regarding the likelihood of efficacy of a therapy comprising administration of an anti-angiogenic drug.

2. The in vitro method according to any one of the aforementioned claims, wherein the prognosis is an assessment regarding the likelihood of efficacy of a combination therapy comprising,
(i) administration of an anti-angiogenic drug, and
(ii) administration of an immune checkpoint inhibitor,
wherein the combination of the two drugs is administered to a patient concomitantly or consecutively.

3. The in vitro method according to any one of the aforementioned claims, wherein the solid tumor is a tumor of a digestive organ.

4. The in vitro method according to any one of the aforementioned claims, wherein the solid tumor is selected from the group consisting of colon cancer, metastatic colon cancer, colorectal cancer or metastatic colorectal cancer, esophageal cancer, metastatic esophageal cancer, gastric cancer and metastatic gastric cancer.

5. The in vitro method according to any one of the aforementioned claims, wherein the sample from the patient has been derived from at least one selected from the group consisting of liver biopsy, lung biopsy, colon biopsy, gastric biopsy, esophageal biopsy and/or colorectal biopsy.

6. The in vitro method according to any one of the aforementioned claims, comprising the step of determining, in the sample, the presence or absence of stroma tissue and/or tumor tissue.

7. The in vitro method according to any one of the aforementioned claims, wherein the anti-angiogenic drug is at least one of
a tyrosin kinase inhibitor, and/or
a binder, inhibitor or antagonist of at least one of VEGF, FGF, PGF, PDGF, VEGFR, FGFR, PGFR or PDGFR.

8. The in vitro method according to claim X, wherein the tyrosin kinase inhibitor is at least one selected from the group consisting of Afatinib, Alectinib, Axitinib, Bosutinib, Cabozantinib, Crizotinib, Dasatinib, Erlotinib, Gefitinib, Gilteritinib, Ibrutinib, Imatinib, Lapatinib, Lenvatinib, Midostaurin, Neratinib, Nilotinib, Nintedanib, Pazopanib, Ponatinib, Regorafenib, Ruxolitinib, Sorafenib, Sunitinib, Tofacitinib, and Vandetanib.

9. The in vitro method according to any one of the aforementioned claims, wherein the immune checkpoint inhibitor is a binder, inhibitor or antagonist of at least one of CTLA-4, PD-1, PD-L1, LAG 3, TIM3, OX40, and/or TIGIT.

10. The in vitro method according to claim X, wherein the antibody is at least one selected from the group consisting of is at least one selected from the group consisting of Ipilimumab (anti-CTLA-4), Nivolumab (anti-PD-1), Pembrolizumab (anti-PD-1), Cemiplimab (anti-PD-1), Spartalizumab (anti-PD-1), Atezolizumab (anti-PD-L1), Avelumab (anti-PD-L1), Durvalumab (anti-PD-L1), Etigilimab (anti-TIGIT), BGB-A1217 (anti-TIGIT) BMS-986207 (anti-TIGIT), AB154 (anti-TIGIT) ASP8374 (anti-TIGIT), MK 7684 (anti-TIGIT), and/or Tiragolumab (anti-TIGIT)

11. The in vitro method according to claim 1, wherein
a) the intratumoral infiltration by macrophages, is determined by at least one of a method comprising an immunoassay or a method comprising PCR
b) the proximity of lymphocytes to tumor cells is determined by a method comprising an immunoassay
c) the proximity of macrophages to tumor cells is determined by a method comprising an immunoassay and/or
d) presence or absence of one or more nearby tertiary lymphoid structures (TLS) is determined by a method comprising an immunoassay.

12. The in vitro method according to claim 1, wherein the intratumoral infiltration by macrophages is determined by means of at least one of
detection of CD163 or CD68 expression in the sample, preferably in the tumor tissue identified therein,
detection of CD163+ cells or CD68+ cells in the tumor, preferably in the tumor tissue identified therein.

13. The method according to any one of the aforementioned claims, wherein the proximity of lymphocytes to tumor cells is determined by determining the distance between lymphocytes and cells characterized by the presence of a tumor marker.

14. The method according to any one of the aforementioned claims, wherein the proximity of macrophages to tumor cells is determined by determining the distance between macrophages and cells characterized by the presence of a tumor marker.

15. The method according to any one of the aforementioned claims, wherein determination of the presence or absence of one or more nearby tertiary lymphoid structures comprises at least one of
detecting the expression, presence or absence of CD3 and CD20 in the sample staining the sample with haematoxyin and Eosin (H&E)
detecting the presence or absence of DC-Lamp⁺ mature dendritic cells (mDCs) and/or CD8⁺ T cells, and/or
detecting the expression, presence or absence of at least one of CD21 (also known as CR2), CD35 (also known as CR1) and CD23 (also known as FcεRII)
detecting the expression, presence or absence of at least one of CD20 and CD19, and/or
detecting the expression, presence or absence of at least one of PNAd.

16. The method according to any one of the aforementioned claims, wherein
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
is indicative for
a high likelihood of improved survival of the patient, and/or
better clinical outcome
in response to anti-angiogenesis therapy and/or immune checkpoint inhibition therapy, based on the therapeutic agents and regimens discussed herein elsewhere.

17. A kit for carrying out a method according to any one of the aforementioned claims, said kit comprising means for detecting at least one of
a) intratumoral infiltration by macrophages, and/or
b) proximity of lymphocytes to tumor cells, and/or
c) proximity of macrophages to tumor cells, and/or
d) presence or absence of one or more nearby tertiary lymphoid structures (TLS)

18. The Kit according to claim X, which kit is suitable for at least one of
Fluorescent in situ hybridization (FISH),
In situ PCR,
Real time PCR, and/or
Reverse transcription PCR/qPCR

19. The Kit according to claim X, which kit comprises at least one immunoligand capable of binding to at least one of CD163, CD68; CD8; Keratin; one or more Cytokeratins; CD3; CD20; DC-LAMP; CD21; CD35; CD23; CD20; CD19, and or PNAd in an immunoassay.

20. The Kit according to claim X, wherein said immunoassay is at least one selected from the group consisting of
IHC (Immunohistochemistry assay)
ICC (Immunocytochemistry assay), and/or
IF (Immunofluorescence assay)

21. An anti-angiogenic drug (in the manufacture of a medicament) for use in the treatment of a patient that suffers from or is at risk of developing a solid tumor, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)

22. A combination of an anti-angiogenic drug and an immune checkpoint inhibitor (in the manufacture of separate coadministrable medicaments) for use in the treatment of a patient that suffers from or is at risk of developing a solid tumor, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
wherein the combination of the two drugs is administered to a patient concomitantly or consecutively.

23. An anti-angiogenic drug (in the manufacture of a medicament) for use in the treatment of a patient that suffers from or is at risk of developing a solid tumor, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)

24. A method of treating a patient that suffers from or is at risk of developing a solid tumor with an anti-angiogenic drug, is provided which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
wherein said method comprises administration of the drug in at least one therapeutically effective dosis.

25. A method of treating a patient that suffers from or is at risk of developing a solid tumor with a combination comprising an anti-angiogenic drug and an immune checkpoint inhibitor, which tumor is characterized by
a) low intratumoral infiltration by macrophages, and/or
b) small distance of lymphocytes to tumor cells, and/or
c) large distance of macrophages to tumor cells, and/or
d) presence of one or more nearby tertiary lymphoid structures (TLS)
wherein said method comprises administration of the combination of the two drugs in at least one therapeutically effective dosis,
and wherein the combination of the two drugs is administered to a patient concomitantly or consecutively.
